(19) United States
(12) Patent Application Publication
(10) Pub. No.: US 2005/0019365 A1
(43) Pub. Date: Jan. 27, 2005

(54) Bioactive surface layer, particularly for medical implants and prostheses

(76) Inventors: Vinzenz M. Frauchiger, Baden (CH); Falko Schlottig, Fulinsdorf (CH); Marcus Textor, Schaffhausen (DE); Beat Gasser, Ittigen (CH)

Correspondence Address: RANKIN, HILL, PORTER & CLARK, LLP, 925 EUCLID AVENUE, SUITE 700, CLEVELAND, OH, 44115-1405, US

(21) Appl. No.: 10/473,768

(22) PCT Filed: Apr. 2, 2001
(86) PCT No.: PCT/CH01/00210

(51) Int. Cl. A61F 2/00

(52) U.S. Cl. 424/423

(57) ABSTRACT

A bioactive surface layer that is particularly suited for medical implants and prostheses. A variable portion of the 0.1 to 50.0 μm thick, porous surface layer includes calcium phosphate phases. The surface layer contains amorphous or nanocrystalline calcium phosphates and the pore density on the surface of the surface layer ranges from 10⁴ to 10⁸ pores/mm². The ratio of Ca/P over the entire surface layer ranges from 0.5 to 2.0. The surface layer has a high solubility so that it can act as a contributor of calcium phosphate for the formation of bone.

The present invention relates to a bioactive surface layer, as specified by claim 1, as well as to a process for fabrication of a bioactive porous surface layer containing calcium phosphate, as specified by Claim 16.

A titanium dental implant is known from U.S. Pat. No. 5,478,237 (ISHIZAWA) which, by means of anodic oxidation of the substrate, is provided with a surface layer containing calcium and phosphate ions, more precisely in the form of hydroxyapatite crystals. A disadvantage of this coating is the fact that the hydroxyapatite crystals are essentially insoluble, so the calcium and phosphate ions cannot be incorporated into the bone. Another disadvantage of this known crystalline coating is its brittleness, which results in risk of exfoliation of the coating. Finally, the disclosed fabrication process is a two-step process, which must be considered a disadvantage.

The present invention intends to remedy this. The aim of the invention is to form a calcium phosphate (CaP)-containing bioactive porous surface layer having highly soluble calcium phosphates so that it can act as a calcium phosphate donor for bone formation.

The invention achieves the indicated aim with a surface layer having the features of Claim 1.

Further advantageous embodiments of the invention are identified in the dependent Claims.

The advantages achieved by the invention are essentially seen to be that, due to the surface layer according to the invention with calcium phosphate phases solubly incorporated in the pore structure, bone can more rapidly grow into the porous support structure. So we do not have just surface impregnation with calcium and phosphate ions, but also a microstructure superimposed on the existing surface structure and achievable by the process according to the invention, depending on the choice of process parameters, with incorporation of amorphous or nanocrystalline—and thus soluble—calcium phosphate phases in the metal oxide layer formed by anodic oxidation using spark discharge.

The process according to the invention has the advantage that it is a fast one-step process and also is suitable for complex geometric shapes.

In a preferred embodiment of the invention, the surface layer additionally contains hydroxyapatite.

The Ca/P ratio over the entire surface layer preferably is within the range from 1.0 to 1.8, i.e., about the same as for TCP [tricalcium phosphate], HA [hydroxyapatite], and calcium-deficient HA. It has been shown that a Ca/P ratio close to that of the natural inorganic bone material hydroxyapatite (1.67) is an optimal Ca/P ratio with regard to the proportion of the layer going into solution.

In a further preferred embodiment of the invention, the amorphous or nanocrystalline calcium phosphates and/or hydroxyapatite make up 1 to 40 volume % of the total surface layer. Better adhesion is provided because the calcium phosphate phase is integrated into the layer grown out of the substrate material.

In a further preferred embodiment of the invention, the surface layer contains from 25 to 95 atomic percent metal oxide, preferably from 30 to 80 atomic percent. It has been shown that the mechanical and chemical stability of the layer matrix is optimal for these percentages of metal oxide.

In a further preferred embodiment of the invention, the metal oxide is in the form of crystals, preferably with crystal size from 10 to 150 nanometers, which is favorable for the solubility of the Ca and P ions.

In a further preferred embodiment of the invention, the metal oxide is titanium dioxide, preferably in the form of anatase or rutile, which have a specific crystal structure.

In a further preferred embodiment of the invention, the Ca²⁺ ions and PO₄³⁻ ions incorporated in the surface layer are distributed over the entire metal oxide layer. In contrast to a purely surface impregnation, such a homogeneous surface composition has the advantage that it enables release of sufficiently large amounts of calcium and phosphate per unit area for bone formation.

In a further preferred embodiment of the invention, the thickness of the surface layer is 0.5 to 10.0 μm. Such a relatively thin layer is advantageous with regard to adhesion, especially under shear loading. It furthermore ensures optimal protection against corrosion and particularly against mechanical abrasion, but without risk of brittle behavior and/or stresses under mechanical loading (especially under shear loading) or thermal loading, as is often the case for layers that are too thick.

In a further preferred embodiment of the invention, the pores of the porous surface layer contain pharmacologically active substances, preferably peptides, growth factors, bone morphogenetic proteins, antibiotics, or anti-inflammatories. The advantage of these additional substances involves their inducing effect. Such active substances provide biological or biochemical support for the bone tissue healing process. The release kinetics (time dependence of the release of the active substance or substances per unit area) in this case can be controlled by the choice of pore size (diameter) and pore volume (pore diameter, pore density).

The surface layer according to the invention is preferably applied to a substrate containing one or more of the elements Ti, Zr, Ta, Nb, Al, V, Mg or alloys thereof. The elements Ti, Zr, Ta, Nb, Al, V, Mg are also called valve metals (see M. M. Lohrengel, “Thin anodic oxide layers on aluminium and other valve metals”, Materials Science and Engineering, R11, No. 6, Dec. 15, 1993). Coating such substrates with the surface layer according to the invention has proven to be particularly advantageous.

The surface layer according to the invention that is applied to these substrates, preferably at least partially, consists of nanocrystalline or microcrystalline oxides or mixed oxides of the metal substrate. This results in an at least osteoconductive, structured, and porous layer and thus a direct bone contact.

The major component of the surface layer preferably consists of nanocrystalline or microcrystalline oxide or mixed oxide of the metal base material, typically in a proportion of 60 to 99 volume %.

In a preferred embodiment, the substrate consists of plastics, preferably polyoxymethylene (POM), polyetheretherketone (PEEK), polyaryletherketone (PAEK), polyetherimide (PEI) or liquid crystal polymer (LCP), polymethylpentene (PMP), polysulfone (PSU), polyethersulfone (PESU or PES), polyethylene terephthalate (PETP), polymethylmethacrylate (PMMA), or ultrahigh molecular weight polyethylene (UHMW-PE), where the substrate is provided with a metallic layer made from valve metals. Application to elastic implants and prostheses is therefore feasible.

The features of Claim 16 characterize the process according to the invention for fabricating a bioactive, porous, and calcium phosphate-containing surface layer on valve metals or alloys thereof as well as valve metal coatings on a substrate.

The following substances are suitable as chelating agents:
a) inorganic carboxylic acids, in particular bidentate or polydentate, or carboxylates thereof, especially: citric acid, tartaric acid, nitriloacetic acid (NTA), ethylenediaminetetraacetic acid (EDTA), 1,2-cyclohexanediaminetetraacetic acid (CDTA), diethylenetriamineacetic acid (DTPA), 2-hydroxyethylethylenediaminetriacetic acid, triethylenetetraaminehexaacetic acid (TTHA);
b) ketones, in particular diketones or polyketones, especially ∃-diketone (CH₃—CO—CH₂—CO—CH₃);
c) organophosphoric acids or organophosphates (with ≧2 phosphate groups);
d) organophosphonic acids or organophosphonates (with ≧2 phosphonate groups);
e) organophosphorous acids or organophosphites (with ≧2 phosphite groups);
   as well as suitable salts of all the above substances.

CaX₆⁴⁻ is especially suitable as an inorganic complexing agent, particularly with X=fluoride.

The concentration of the complexing agent advantageously is 0.06 to 0.24 mol/L. Calcium bis(dihydrogen phosphate) is advantageously used as a phosphate compound, typically with a concentration from 0.01 to 0.05 mol/L. Water-soluble calcium compounds (preferably calcium acetate) are preferably used with a concentration from 0.03 to 0.15 mol/L.

Hydroxide compounds are suitable as a basic additive, preferably sodium or potassium hydroxide, typically with a concentration from 0.5 to 1.5 mol/L.

In a preferred embodiment of the coating process, the parameters of the anodizing process (voltage, current, frequency, coating time, bath geometry, etc.) are selected so that the layer is formed by reaction between the substrate and the electrolyte, where the spark discharge results in partial recrystallization of the already formed layer. The temperature of the electrolyte during the coating process is conveniently 10° C. to 90° C., preferably 20° C. to 75° C.

Preferably substrates made from the elements Ti, Zr, Ta, Nb, Al, V, Mg, or alloys thereof, or else barrier-forming metal coatings on any substrates of any shape and surface condition, are coated all over or partially. The surface topography or morphology is advantageously manipulated by chemical and/or mechanical pretreatments of the starting surface, where the chemical pretreatment can be an etching process and the mechanical pretreatment can be a blasting process.

The invention and refinements of the invention are explained in greater detail in the following, with the help of an exemplary embodiment.

### EXAMPLE

A cylindrical disk made from pure titanium (height 1 mm, diameter 7 mm), in the following electrolyte solution at a temperature of 25° C.:

| | |
|---|---|
| 10.5 g | calcium dihydrogen phosphate |
| 22 g | calcium diacetate |
| 74 g | ethylenediaminetetraacetic acid, disodium salt |
| 13 g | sodium hydroxide |

was brought up to 1 liter total volume with high-purity water,
was galvanostatically coated with a current of 80 mA for 90 seconds.

The light gray coating obtained had the following concentration ratios, in atomic percent:
Ca/Ti=1.05, P/Ti=0.83, and Ca/P=1.27.

1. A bioactive surface layer, in particular for medical implants and prostheses, wherein:
A) a variable proportion of the surface layer consists of calcium phosphate phases;
B) the surface layer contains a proportion of 25 to 95 atomic percent metal oxide of the metallic base material;
C) the thickness of the layer is between 0.1 and 50.0 μm;
D) the surface layer is porous;
E) the surface layer contains amorphous or nanocrystalline calcium phosphates; and
F) the Ca/P ratio over the entire surface layer is in the range between 0.5 and 2.0;
wherein:
G) the Ca-ions and P0₄-ions embedded in the surface layer are distributed over the entire metal oxide layer;
H) the pore density on the surface of the surface layer is between 10⁴ and 10⁸ pores/mm²; and
I) the amorphous or nanocrystalline calcium phosphates as well as any possible hydroxyapatite portions make up 1 to 40 volume % of the total surface layer.

2. The surface layer as recited in claim 1, wherein the surface layer consists of hydroxyapatite.

3. The surface layer as recited in claim 1, wherein the surface layer additionally contains hydroxyapatite.

4. The surface layer as recited in claim 1, wherein the pore density is between 10⁵ and 10⁷ pores/mm².

5. The surface layer as recited in claim 1. wherein the Ca/P ratio over the entire surface layer is between about 1.0 and 1.8.

6. The surface layer as recited in claim 1, further comprising contains a proportion of 30 to 80 atomic percent metal oxide.

7. The surface layer as recited in claim 6, wherein the metal oxide is in the form of crystals, having a crystal size of 10 to 150 nanometers.

8. The surface layer as recited in claim 6, wherein the metal oxide is titanium oxide.

9. The surface layer as recited in claim 8, wherein the titanium oxide is in the form of anatase or rutile.

10. The surface layer as recited in claim 1 wherein the thickness of the layer is between 0.5 μm and 10.0 μm.

11. The surface layer as recited in claim 1, wherein the pores of the porous surface layer contain pharmacologically active substances, preferably peptides, growth factors, bone morphogenetic proteins, antibiotics, or anti-inflammatories.

12. A substrate having a surface layer as recited in claim 1, wherein the substrate contains one or more of the elements Ti, Zr, Ta, Nb, Al, V, Mg (valve metals) or alloys thereof.

13. The substrate as recited in claim 12, wherein the surface layer at least partially consists of nanocrystalline or microcrystalline oxides or mixed oxides of the metal substrate.

14. The substrate as recited in claim 13, wherein the main component of the surface layer consists of the nanocrystalline or microcrystalline oxide or mixed oxide of the metal substrate, preferably in a proportion of 60 to 99 volume %.

15. A substrate having a surface layer as recited in claim 1, wherein the substrate consists of plastics, preferably polyoxymethylene (POM), polyetheretherketone (PEEK), polyaryletherketone (PAEK), polyetherimide (PEI) or liquid crystal polymer (LCP), polymethylpentene (PMP), polysulfone (PSU), polyethersulfone (PESU or PES), polyethylene terephthalate (PETP), polymethylmethacrylate (PMMA), or ultrahigh molecular weight polyethylene (UHMW-PE), where the substrate is provided with a metallic layer made from valve metals.

16. A method for fabricating a bioactive, porous, and calcium phosphate-containing surface layer on valve metals or alloys thereof, as well as valve metal coatings on a substrate, where a substrate to be coated is anodically exposed to an aqueous electrolyte containing calcium and phosphate ions, which are to be embedded into the forming layer, and where an anodic plasma-chemical surface modification takes place in the electrolyte by spark discharge using direct current voltage or direct current voltage pulses and time variation of the voltage wherein:
A) the aqueous electrolyte is brought to a pH-value larger than or equal to 9, using calcium and phosphate additives, and contains at least the following components:
B1) one or more organic chelating agents or inorganic complexing agents in a concentration range between 0.01 and 6.00 mol/L;
B2) one more phosphate compounds in a concentration range between 0.01 and 6.00 mol/L, preferably between 0.01 and 0.05 mol/L;
B3) one or more water-soluble calcium compounds for arriving at the desired calcium/phosphate ratio of 0.01 to 6.00 mol/L; and
B4) one or more basic additives in a concentration range between 0.01 and 6.00 mol/L for arriving at the desired pH-value.

17. The method as recited in claim 16, wherein the chelating agent is an inorganic carboxylic acid, preferably bidentate or polydentate, or carboxylates thereof.

18. The method as recited in claim 17, wherein the inorganic carboxylic acid is selected from the following group: citric acid, tartaric acid, nitriloacetic acid (NTA), ethylenediaminetetraacetic acid (EDTA), 1,2-cyclohexanediaminetetraacetic acid (CDTA), diethylenetriamineacetic acid (DTPA), 2-hydroxyethylethylenediaminetriacetic acid, triethylenetetraaminehexaacetic acid (TTHA).

19. The method as recited in claim 16, wherein the chelating agent is a ketone, preferably a diketone or a polyketone.

20. The method as recited in claim 19, wherein the diketone is ∃-diketone (CH₃—CO—CH₂—CO—CH₃).

21. The method as recited in claim 16, wherein the chelating agent is an organophosphoric acid or an organophosphate, with preferably ≧2 phosphate groups.

22. The method as recited in claim 16, wherein the chelating agent is an organophosphonic acid or an organophosphonate, with preferably ≧2 phosphonate groups.

23. The method as recited in claim 16, wherein the chelating agent is an organophosphorous acid or an organophosphite, with preferably ≧2 phosphite groups.

24. The method as recited in claim 16, wherein the chelating agent is a salt.

25. The method as recited in claim 16, wherein the inorganic complexing agent comprises CaX6⁴⁻, with X=fluoride.

26. The method as recited in claim 16, wherein the complexing agent has a concentration of 0.06 to 0.24 mol/L.

27. The method as recited in claim 16, wherein the phosphate compound is calcium bis(dihydrogen phosphate).

28. The method as recited in claim 16, wherein the phosphate compound has a concentration of 0.01 to 0.05 mol/L.

29. The method as recited in claim 16, wherein the water-soluble calcium compound is calcium acetate.

30. The method as recited in claim 16, wherein the water-soluble calcium compound has a concentration of 0.03 to 0.15 mol/L.

31. The method as recited in clam 16, wherein the basic additive is a hydroxide compound, preferably sodium or potassium hydroxide.

32. The method as recited in claim 16, wherein the basic additive has a concentration of 0.5 to 1.5 mol/L.

33. The method as recited in claim 16, wherein the parameters of the anodizing process (voltage, current, frequency, coating time, bath geometry, etc.) are selected so that the layer is formed by reaction between the substrate and the electrolyte, where the spark discharge results in partial recrystallization of the already formed layer.

34. The method as recited in claim 16, wherein the temperature of the electrolyte during the coating process is 10° C. to 90° C., preferably 20° C. to 75° C.

35. The method as recited in claim 16, wherein the substrates made from the elements Ti, Zr, Ta, Nb, Al, V, Mg, or alloys thereof, or else barrier-forming metal coatings on any substrates of any shape and surface condition, are coated all over or partially.

36. The method as recited in claim 16, wherein the surface topography or morphology is manipulated by chemical and/or mechanical pretreatments of the starting surface.

37. The method as recited in claim 36, wherein the chemical pretreatment is an etching process.

38. The method as recited in claim 36, wherein the chemical pretreatment is a blasting process.
